(19) United States
(12) Patent Application Publication
(10) Pub. No.: US 2009/0030399 A1
(43) Pub. Date: Jan. 29, 2009

(54) Drug Delivery Device and Method

(76) Inventor: Kamshad Raiszadeh, La Jolla, CA (US)

Correspondence Address: BROMBERG & SUNSTEIN LLP, 125 SUMMER STREET, BOSTON, MA, 02110-1618, US

(21) Appl. No.: 12/178,544

(22) Filed: Jul. 23, 2008

Related U.S. Application Data
(60) Provisional application No. 60/951,263, filed on Jul. 23, 2007.

(51) Int. Cl. A61M 5/142; A61F 2/44; A61B 17/70; A61M 31/00

(52) U.S. Cl. 604/506; 623/17.12; 604/288.04; 604/151; 606/246

(57) ABSTRACT

The present invention is directed to a medical device and method for delivering a drug. The medical device includes a deformable body configured to be implanted between a first vertebra and a second vertebra for providing shock absorption and stabilization of the vertebras. The deformable body comprises an impermeable inner layer forming an interior volume and an outer layer at least a portion of which is permeable. The outer layer is at least partially outside the inner layer and forms an exterior volume between the inner and outer layers. The medical device further includes a drug reservoir connected to the deformable body and in fluid communication with the exterior volume. The medical device is capable of delivering the drug from the reservoir into the exterior volume and releasing the drug through the permeable portion of the outer layer.

The present application claims the benefit of U.S. Application Ser. No. 60/951,263, entitled “MEDICAL DEVICES AND RELATED METHODS” filed Jul. 23, 2007, which application is incorporated herein by reference in its entirety.

### TECHNICAL FIELD

The present invention relates to medical devices and related methods, and more particularly to drug delivery devices and related methods.

### BACKGROUND ART

The human spine includes a series of vertebras. Adjacent vertebras are separated by an anterior intervertebral disc and two posterior facets joints. Together, the disc and facet joints create a spinal motion segment that allows the spine to flex, rotate, and bend laterally. The intervertebral disc also functions as a spacer and a shock absorber. As a spacer, the disc provides proper spacing that facilitates the biomechanics of spinal motion and prevents compression of spinal nerves. As a shock absorber, the disc allows the spine to compress and rebound during activities, such as jumping and running, and resists the axial pressure of gravity during prolonged sitting and standing.

Sometimes, the disc and facets can degenerate, for example, due to the natural process of aging, and produce large amounts of pain. A number of procedures have been developed to treat degeneration of the spinal motion segment. For example, the vertebras directly adjacent to the disc can be fused together, the disc can be removed by discectomy procedure, or the disc can be replaced by disc arthroplasty. Yet, many of these procedures are also accompanied by large amounts of post-operative pain.

### SUMMARY OF THE INVENTION

Embodiments of the present invention are directed to a medical device and a method for delivering a drug to a spinal segment and for providing support to the spinal segment. In some embodiments, the drug may provide post-operative pain relief. One embodiment of the medical device includes a deformable body configured to be implanted between a first vertebra and a second vertebra for providing shock absorption and stabilization of the vertebras. The deformable body comprises an impermeable inner layer forming an interior volume and an outer layer at least a portion of which is permeable. The outer layer is at least partially outside the inner layer and forms an exterior volume between the inner and outer layers. The medical device further includes a drug reservoir connected to the deformable body and in fluid communication with the exterior volume. The medical device is capable of delivering the drug from the reservoir into the exterior volume and releasing the drug through the permeable portion of the outer layer.

In some embodiments, at least a portion of the outer layer is a porous membrane. Additionally or alternatively, the outer layer may include a microvalve. The outer layer may also be coextensive with the inner layer. In another embodiment, the outer layer and inner layer are adhered together in order to form channels that allow drug delivery to selected areas of the implant site. With respect to the inner layer, in some embodiments, the inner layer may be reinforced with a mesh material.

In other embodiments the medical device may include a catheter having a lumen. The catheter connects the drug reservoir to the deformable body. The lumen is configured to deliver a drug from the drug reservoir to the exterior volume. In a further embodiment, a pump may be in fluid communication with the exterior volume for delivering the drug from the drug reservoir to the exterior volume. The pump may be configured to be implanted subcutaneously and be remotely controllable. The medical device may also include a filling valve for modulating the drug in the drug reservoir. For example, the filling valve may be an infusion port.

In another embodiment of the medical device, the medical device includes a deformable body configured to be implanted between a first vertebra and a second vertebra for providing shock absorption and stabilization of the vertebras. The deformable body comprises a deformable exterior wall and a deformable interior wall forming an interior volume. The medical device also includes a channel formed between the exterior and interior walls. The channel is in fluid communication with an opening formed in the exterior wall of the deformable body. The medical device further include a drug reservoir connected to the deformable body and in fluid communication with the channel. The medical device is capable of delivering the drug from the drug reservoir into the channel and releasing the drug through the opening in the exterior wall.

In some embodiments the medical device includes a plurality of openings and/or a plurality of channels. The openings and/or channels may have different dimensions. In other embodiments, the medical device includes at least one valve in fluid communication with the channel for modulating the release of the drug through at least one opening. For example, the valve may be a microvalve located in the exterior wall.

In other exemplary embodiments, the medical device includes a catheter having a lumen. The catheter connects the drug reservoir to the deformable body and the lumen is configured to deliver a drug from the drug reservoir through the opening. In a further embodiment, a pump may be in fluid communication with the channel for delivering the drug from the drug reservoir to the channel and through the opening. The pump may be configured to be implanted subcutaneously and be remotely controllable. As in other embodiments, the medical device may also include a filling valve for modulating the drug in the drug reservoir. For example, the filling valve may be an infusion port.

In embodiments of the medical device that include an interior volume, the medical device may include a filling valve located outside the deformable body and in fluid communication with the interior volume. The filling valve is configured to allow for post-operative addition or removal of fluid in the interior volume. The filling valve may be, for example, an infusion port. Further, the medical device may include a reservoir for modulation of liquid within the interior volume. The reservoir may be connected to the deformable body and in fluid communication with the interior volume.

Other embodiments of the medical device that include an interior volume may include an extradiscal portion spaced from the deformable body. The extradiscal portion is connected to the deformable body and is in fluid communication with the interior volume of the deformable body. This extradiscal portion may be expandable.

In related embodiments, the medical device may include a first connector for attaching the extradiscal portion to a first portion of a spinal segment. The first connector may be a pedicle screw. A further embodiment may include a second connector for attaching the extradiscal portion to a second portion of the spinal segment. The first portion and second portion of the spinal segment may be, respectively, a first vertebra and a second vertebra. In another embodiment, the first portion and second portion of the spinal segment may be, respectively, a first spinous process and a second spinous process.

In some embodiments, the extradiscal portion may be configured to connect to the first and second spinal segments so that movement of the spinal segments and pressure on the intradiscal portion applies hydraulic pressure to the extradiscal portion. In other embodiments, the extradiscal portion may include a piston.

Embodiments of the present invention are also directed to a method for delivering a drug. The method includes providing a medical device having a deformable body configured to be implanted between a first vertebra and a second vertebra for providing shock absorption and stabilization of the vertebras, and having a drug reservoir connected to the deformable body. The device is capable of delivering a drug from the drug reservoir into the deformable body and releasing the drug through the deformable body into an implant site. The method also includes positioning the deformable body between two vertebras and, after positioning the deformable body between the two vertebras, adding biocompatible fluid into the deformable body. The method further includes positioning the drug reservoir in the body, adding a drug to the drug reservoir, and retaining in the body post-operatively the drug reservoir and the deformable body between the two vertebras. In some embodiments, the deformable body and the drug reservoir are positioned in the body from a posterior approach.

The method may also include removing at least a portion of a disc between the two vertebras. A test balloon may be expanded between the vertebras. A contrast agent may be added into the test balloon.

The method may further include delivering the drug from the drug reservoir to the deformable body and/or pumping the drug from the reservoir to the deformable body. In some embodiments, pumping the drug to the deformable body may be controlled remotely and post-operatively. The drug delivered to the deformable body may be an anesthetic. The rate of drug delivery may be changed. Further, the rate of delivery may be a function of pain experienced by the patient. In other embodiments, the drug in the drug reservoir may be modulated post-operatively.

The method may further include modulating the biocompatible fluid in the deformable body post-operatively. The biocompatible fluid may be modulated from a posterior approach through a filling valve in fluid communication with the deformable body. In a related embodiment, the device may include an expandable extradiscal portion in fluid communication with the deformable body, and the method may further include positioning the expandable extradiscal portion spaced from the vertebras. Additionally or alternatively, the expandable extradiscal portion may be positioned posterior of the vertebras.

The device and method are not limited to use with the spine. In related embodiments, the medical device can be implanted within other structures in the body. For example, a deformable body can act as a shock absorber simulating cartilage within a joint, such as a knee or a hip. Pain medication can be delivered into the joint for pain relief and mechanical stabilization can be afforded by the deformable body. A deformable body can be used in the intercostal area between the ribs for treatment of scoliotic deformities. Also, traumatic defects in muscle or bone can be filled by a deformable body whose volume can be adjusted via a fluid reservoir. This device can be useful, for example, if there are contractions of the skin or soft tissues. The device may gradually lengthen the tissues by increasing the volume within the deformable body.

### BRIEF DESCRIPTION OF THE DRAWINGS

The foregoing features of the invention will be more readily understood by reference to the following detailed description, taken with reference to the accompanying drawings, in which:

FIG. 1 is a schematic view of an embodiment of a medical device between two vertebras;

FIG. 2 is a detailed view of the area 2 depicted in FIG. 1;

FIG. 3 is a cross-sectional view of FIG. 2, taken along line 3-3;

FIG. 4 is a cross-sectional view of FIG. 2, taken along line 4-4;

FIG. 5 depicts a method of implanting a medical device;

FIG. 6A is an illustration of the L5 and S1 vertebras;

FIG. 6B is schematic view of a portion of an embodiment of a medical device;

FIG. 6C is schematic view of the medical device shown in FIG. 6B implanted between the L5 and S1 vertebras;

FIG. 7 is a schematic view of an embodiment of a medical device;

FIG. 8 is a cross-sectional view of an embodiment of a valve in a closed position;

FIG. 9 is a cross-sectional view of the valve depicted in FIG. 8 in an opened position;

FIG. 10 is detailed view of an embodiment of a medical device;

FIG. 11 is another detailed view of the medical device shown in FIG. 10;

FIG. 12 is cross-sectional diagram of an embodiment of a medical device;

FIG. 13 is a schematic view of a portion of an embodiment of a medical device between two vertebras;

FIG. 14 is a side view of the medical device shown in FIG. 13; and

FIG. 15 is a posterior view of the medical device shown in FIG. 13.

### DETAILED DESCRIPTION OF SPECIFIC EMBODIMENTS

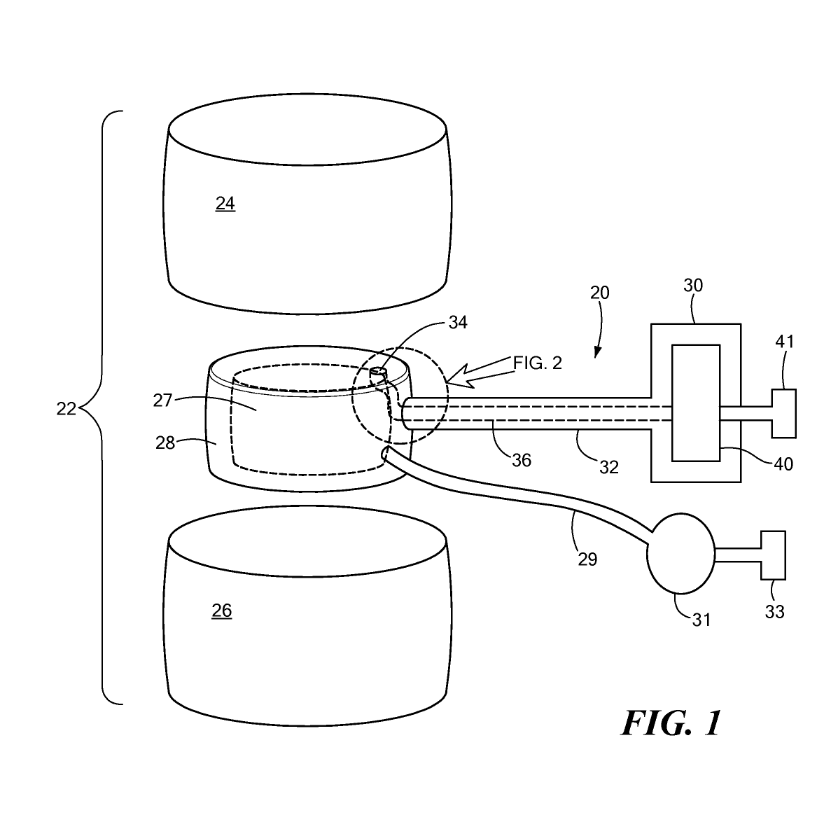

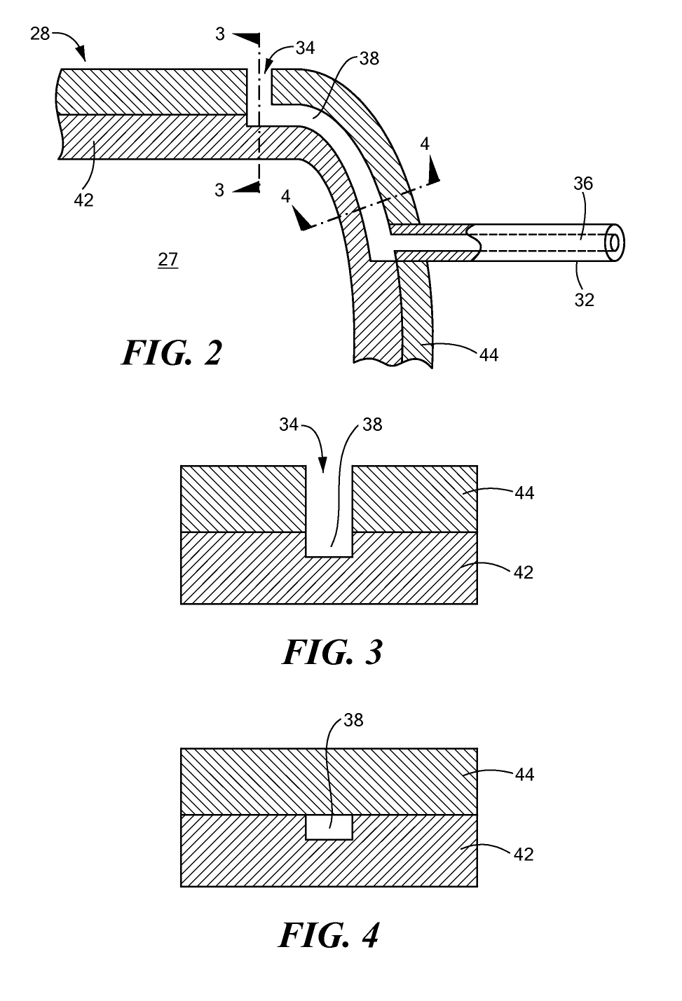

Referring to FIG. 1, a medical device 20 is shown implanted along a spinal segment 22 between a superior vertebra 24 and an inferior vertebra 26. Medical device 20 includes a deformable body 28 (e.g., a balloon) and an implantable pump 30 that is connected to the deformable body by a hollow catheter 32 having a lumen 36. Deformable body 28 includes an exterior wall and an interior wall. The interior and exterior walls may be deformable. The interior wall forms an interior volume 27 filled with a biocompatible fluid, such as saline. Interior volume 27 is in fluid communication via a hollow conduit 29 with an implantable fluid reservoir 31 that is also filled with the biocompatible fluid. Fluid reservoir 31 has a filling valve 33 for changing the amount of fluid in the fluid reservoir. The fluid reservoir 31 and/or the filling valve 33 may be implanted subcutaneously or may be located partially or wholly above the skin. Referring also to FIGS. 2, 3, and 4, at one end, lumen 36 is in fluid communication with a channel 38 between the exterior and interior walls and an opening 34 formed in the exterior wall of deformable body 28. The channel 38 is in fluid communication with the opening 34. At the other end, lumen 36 is in fluid communication with a pump 30, specifically a drug reservoir 40 associated with the pump and containing a drug (such as an anesthetic). The pump 30 and/or the drug reservoir 40 may be located subcutaneously, or partially or wholly above the skin. Drug reservoir 40 has a filling valve 41 for changing the amount of drug in the drug reservoir. The filling valve 41 may also be located subcutaneously, or partially or wholly above the skin. For convenience and clarity, the specification may refer to filling valves, reservoirs, and pumps as “subcutaneous,” however, the use of the term “subcutaneous” does not limit other embodiments, wherein the filling valves, reservoirs, and pumps may be located wholly or partially above the skin. Pump 30 is capable of delivering the drug from drug reservoir 40, through lumen 36 and channel 38, and out opening 34, where the drug can provide a desired effect to a desired area (e.g., within the spinal segment). The opening 34 and the channel 38 may be configured so as to deliver the drug to selected areas in the implant site (e.g., posteriorly and/or anteriorly). The drug can include an anesthetic that is used to alleviate pain originating from nerves located between vertebras 24, 26, such as in the disc space. Alternatively or additionally, a narcotic medication, such as morphine and/or fentanyl, can be delivered. By delivering the drug directly to the source of pain, vis-a-vis systemically, the pain can be quickly addressed and/or the drug dosage can be reduced, which can lower the occurrence of unwanted side effects.

Deformable body 28 is generally configured to be placed, wholly or partially, between two vertebras to serve as a spacer and/or a shock absorber. For example, deformable body 28 can prevent spinal nerves from pinching, and/or can resiliently cushion compressive forces of the motion segment in which it is introduced. Fluid reservoir 31 can be used to control or regulate the amount of fluid in deformable body 28. For example, by adding more fluid to fluid reservoir 31, deformable body 28 can be expanded to distract the vertebral bodies, resulting in decompression of previously compressed nerves. Compressive forces can occur during activities such as running or jumping, or during prolonged periods of sitting or standing.

Furthermore, deformable body 28 is capable of mimicking an intervertebral disc to allow spinal segment 22 to move normally (e.g., by filling the space occupied by the disc and restore the height provided by the disc). In particular, hydraulic pressure is used from the fluid filled in deformable body 28 to stabilize spinal segment 22 during motion. For example, when the patient bends or flexes forward, this movement can compress deformable body 28, thereby transferring fluid by hydraulic pressure from the deformable body 28 to fluid reservoir 31 via conduit 29. Fluid reservoir 31 can expand as a result of the additional fluid. As described below, in some embodiments, a medical device includes multiple extradiscal reservoirs. As a result, when the patient bends or flexes backward, this movement can compress the extradiscal portions, thereby transferring fluid by hydraulic pressure from the extradiscal portions to deformable body 28, which can expand as a result of the additional fluid. Similarly, when the patient rotates or bends laterally, fluid from one of extradiscal portions can flow to and expand deformable body 28 and/or the other extradiscal portion. Thus, the medical device system is capable of allowing spinal segment 22, such as a lumbar spinal segment, to move, for example, flex, rotate, and/or bend, relatively naturally while still maintaining mechanical integrity and stability. As shown, conduit 29 and fluid reservoir 31 are separated from catheter 32 and pump 30, but in some embodiments, the fluid reservoir and the pump are positioned within the same implantable housing, and the conduit and the catheter are formed as a catheter having multiple lumens.

Although not depicted in FIG. 1, in further embodiments, the medical device 20 may include a smart controller with a processor. The smart controller may be located within the same implantable housing as the pump 30, drug reservoir 40, and/or fluid reservoir 31. The smart controller may be used to control the drug pump 30 and/or any other valves involved in drug delivery to the spinal segment (e.g., the microvalves explained below). The smart controller may also be used to control the fluid reservoir 31 and/or any other pumps or valves involved in the movement of fluid from the fluid reservoir 31 through conduit 29 into deformable body 28. Although not depicted, medical device 20 may also include a fluid pump for transferring fluid between fluid reservoir 31 and deformable body 28. The smart controller may be in communication with this fluid pump, thereby allowing the smart controller to modulate the transfer of fluid between the fluid reservoir 31 and the deformable body 28.

In some embodiments, the smart controller may be in communication with one or more load sensors located on or within the deformable body 28 (e.g., through a feed back loop). Based on feedback from the load sensors, the smart controller may deliver the drug to the spinal segment as a function of the load. For example, if a patient experiences a fall, the increased load on the spine due to the fall is registered by the load sensors, and the smart controller reacts by delivering a controlled dose of an anesthetic drug. The smart controller may also react by increasing amount of fluid within the deformable body to provide additional support, or by decreasing the amount of fluid to relieve pressure in the spinal segment. In other embodiments, the smart controller may be in communication with one or more pressure and/or strain sensors located on or within the deformable body that register a change in the amount of fluid within the deformable body 28. Based on feedback from the sensors, the smart controller can deliver the drug to the spinal segment as a function of the amount of fluid in the deformable body and/or the change in the amount of fluid in the deformable body 28. Or vice-versa, the controller may modulate the amount of fluid in the deformable body 28 based on the dosage or amount of drug delivered to the spinal segment.

Deformable body 28 can include (e.g., be formed of) a biocompatible flexible material that can be expanded by the addition of fluid into the deformable body. The flexibility of the material may allow spinal segment 22 to move relatively naturally. In some embodiments, biocompatible materials used in deformable body 28 are also capable of withstanding stresses applied to an intervertebral disc (e.g., stress forces of greater than 400 pound force/square inch (psi) during lifting and 40-70 psi during normal activities). The material can be implanted in the patient for an extended period of time (e.g., for several years or more). In certain embodiments, deformable body 28 is implanted permanently, and need not be removed. In certain embodiments, lumen 36 can be re-cannulated when disconnected from reservoir 40. An exchange implant can then be deployed.

Examples of flexible biocompatible materials that can be used to form deformable body 28, as well as fluid reservoir 31 and conduit 29, include pure polymers, polymer blends, and copolymers. Examples of polymers include nylon, silicon, latex, and polyurethane. For example, the elongated member can be made from materials similar or identical to the high performance nylon used in the RX Dilation Balloons from Boston Scientific (Natick, Mass.), wherein the material is reinforced or thickened to withstand the forces described herein. Other flexible biocompatible materials include block co-polymers such as castable thermoplastic polyurethanes, for instance, those available under the trade names CARBOTHANE (Thermedics) ESTANE (Goodrich), PELLETHANE (Dow), TEXIN (Bayer), Roylar (Uniroyal), and ELASTOTHANE (Thiocol), as well as castable linear polyurethane ureas, such as those available under the tradenames CHRONOFLEX AR (Cardiotech), BIONATE (Polymer Technology Group), and BIOMER (Thoratec). Other examples are described, for example, in M. Szycher, “Biostability of polyurethane elastomers: a critical review”, J. Biomater. Appl. 3(2):297-402 (1988); A. Coury, et al., “Factors and Interactions Affecting the Performance of Polyurethane Elastomers in Medical Devices”, J Biomater. Appl. 3(2):130-179 (1988); and Pavlova M, et al., “Biocompatible and Biodegradable Polyurethane Polymers,” Biomaterials 14(13):1024-1029 (1993), all of which are incorporated herein in their entirety.

In some embodiments, deformable body 28 includes: (i) multiple layers of the same or different materials, (ii) reinforcing materials, and/or (iii) sections of varied thickness (e.g., designed to withstand the forces described herein). Methods for shaping and forming flexible biocompatible materials, such as casting, co-extrusion, blow molding, and co-blowing techniques, are described, for example, in “Casting”, pp. 109-110, in Concise Encyclopedia of Polymer Science and Engineering, Kroschwitz, ed., John Wiley & Sons, Hoboken, N.J. (1990); U.S. Pat. Nos. 5,447,497; 5,587,125; 5,769,817; 5,797,877; 5,620,649; and International Patent Application No. WO002613A1, all of which are incorporated herein in their entirety. In some embodiments, deformable body 28 includes a coextensive outer layer that can contain the deformable body in the event of rupture, act against long term effects like creep of the deformable body, and restrict expansion of the deformable body. Examples of the outer layer include woven mesh material found in, for example, Raymedica Prosthetic Disc Nucleus® (PDN) device, the SpineMedica SaluDisc™, and/or the Artelon® CMC Spacer system.

Channel 38 and opening 34 can be formed in deformable body 28 using, for example, laser ablation techniques. Referring again to FIGS. 2, 3 and 4, deformable body 28 can be formed by starting with a first biocompatible material 42 as described above and having the general shape as the deformable body. A groove can then be formed in the biocompatible material using laser ablation or a mechanical technique, such as scoring. Next, the groove can be filled with a sacrificial material that can be later selectively removed without affecting the biocompatible material. For example, the sacrificial material can be selectively dissolved with a solvent that does not react with the biocompatible material, or the sacrificial material can have a melting point that is lower than a melting point of the biocompatible material. After the groove is filled, a second biocompatible material 44 is formed over (e.g., coextensively and adhered to) first compatible material 42 and the sacrificial material, for example, by molding or casting. Second biocompatible material 44 can have the same or a different composition as that of first compatible material 42. Opening 34 (e.g., one end of the groove) and a second opening (e.g., at the other of the groove) can then be formed, for example, by laser ablating over the previously formed groove and through second biocompatible material 44. Laser ablation is described, for example, in Weber, U.S. Pat. No. 6,517,888. Next, channel 38 can be formed by removing the sacrificial material from the groove. For example, the sacrificial material can be dissolved in a solvent or melted, and removed from the groove, for example, by applying air pressure to an opening. After opening 34 and channel 38 are formed, deformable body 28 can be connected to catheter 32, for example, by melt bonding and/or using an adhesive, so that lumen 36 of the catheter is in fluid communication with opening 34 and channel 38.

While deformable body 28 is described as having one opening 34 and channel 38, in other embodiments, the deformable body includes multiple openings and/or channels in fluid communication with lumen 36. The multiple openings and/or channels can be used to deliver the drug to one or more specific sites. For example, the opening(s) and/or channel(s) can direct the drug posteriorly in the disc. The openings and/or channels can have the same dimensions (e.g., diameters) or different dimensions to control the amount of the drug that is delivered.

Catheter 32 is generally an elongated, hollow tube. The Catheter 32 can include (e.g., be formed of) one or more biocompatible material described above for deformable body 28. Pump 30 is generally an implantable (e.g., subcutaneously) device capable of delivering a drug from a reservoir to deformable body 28. In some embodiments, pump 30 can be remotely controlled, for example, to deliver a bolus dose and/or to change the frequency of doses. An example of pump 30 is an intrathecal pain pump, commercially available from Medtronic, Inc. As shown in FIG. 1, reservoir 40 is contained in pump 30, but in other embodiments, the reservoir and the pump are distinct components that are interfaced so that the pump can control delivery of the contents (e.g., a drug) of the reservoir. More than one pump and/or reservoir can be used, for example, to deliver different drugs to deformable body 28.

The drug contained in reservoir 40 can be any compound used to treat the body. Examples of the drug include anesthetics, such as morphine, lidocaine, Marcaine®/Sensorcaine (bupivacaine), and zidocaine. More than one drug can be delivered by medical device 20.

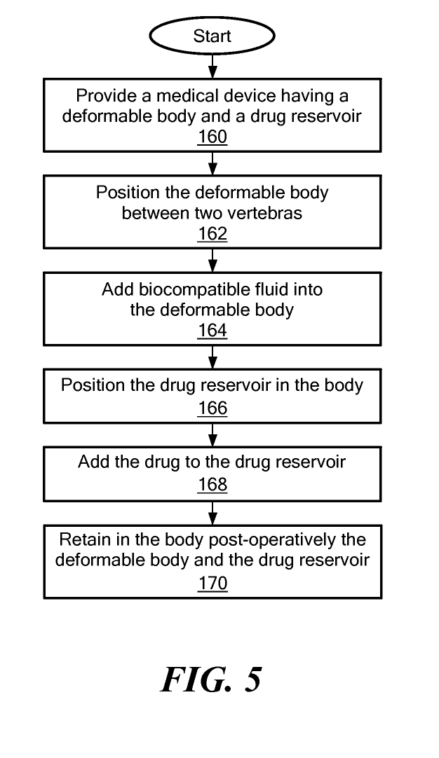

Turning now to a method of implanting medical device 20. The following method may be employed with any of the disclosed embodiments of the medical device (e.g., 20, 60, 200). FIG. 5 depicts a method for implanting the medical device. The method in overview includes first forming a disc space by, for example, removing at least a portion of the nucleus of an intervertebral disc. Next, the disc space is measured. A test balloon can be inserted into the disc space to determine the size of the disc space. The medical device 20, as described above, is provided 160. An appropriately-sized deformable body 28 is then inserted into the disc space and positioned between the two vertebras 162. The deformable body 28 is then filled with a biocompatible fluid 164. In some embodiments the catheter 32, pump 30, conduit 29, and reservoir 31 are connected to the deformable body 28 and the deformable body is filled via the reservoir 31. In other embodiments the deformable body is filled via, for example, a valve and a filler tube (not shown). Then, the catheter 32, pump 30, conduit 29, and reservoir 31 are connected to the deformable body and positioned in the desired places in the body 166. Then, the drug is added to the drug reservoir 168 and the deformable body and the drug reservoir are retained in the body post-operatively 170.

More specifically, the method of implanting medical device 20 includes removing at least a portion of an intervertebral disc to prepare the implantation site. Typically, a spinal segment includes a disc, which includes a nucleus surrounded by an annulus, located between superior vertebra 24 and inferior vertebra 26. A unilateral or bilateral spinal discectomy can be performed, for example, with a standard laminectomy or with a minimally invasive lumbar incision posterior to the patient's spine, to remove at least a portion of or as much as possible (e.g., all) of the nucleus to form a disc space. In some embodiments, a portion of or all of the annulus is also removed by either a laminectomy or a minimally invasive procedure. Discectomy and laminectomy procedures are described, for example, in Bridwell et al., Eds., “The Textbook of Spinal Surgery, Second Edition,” Lippincott-Raven, Philadelphia, Pa. (1997), which is incorporated herein by reference in its entirety.

After the disc space is formed, the disc space is measured. A test balloon is inserted into the disc space to determine the position and volume of the disc space. The position and volume of the disc space can be used to determine one or more of the following: (i) that the desired disc space was formed, (ii) the desired disc height to be restored, and (iii) the size and type of deformable body 28 that can be used. The test balloon can be inflated with, for example, a fluid containing a contrast agent (such as an omnipaque-containing material) and detected using intra-operative fluoroscopy.

After the test balloon is withdrawn from the disc space, deformable body 28 is placed into the disc space. Biocompatible fluid is added into the deformable body 28 via, for example, a valve and a filler tube (not shown). The amount of fluid added into deformable body 28 can be a function of disc height, and fluid pressure. The amount of fluid added can be modulated after the above index procedure depending on the patient's pain response. For example, after the index procedure to insert deformable body 28, the patient is ambulated and allowed to perform regular activities. The pressure in deformable body 28 can then be changed incrementally post-operatively and over time via a subcutaneous filling valve (e.g., in fluid communication with fluid reservoir 31) to further stabilize the spinal segment if there is pain. In some embodiments, fluid is added until normal disc height is restored, normal motion is restored, and/or pain is decreased. When the desired amount of fluid has been added into deformable body 28, it is connected to fluid reservoir 31 via conduit 29. The amount of fluid within fluid reservoir 31 and deformable body 28 can be changed percutaneously and post-operatively via filling valve 33. In some embodiments, deformable body 28 is partially inflated by, for example, containing a predetermined amount of fluid, prior to implantation to ease handling and inserting of device 20.

Catheter 32 and pump 30 can be positioned, for example, in the subcutaneous space in the flank or abdomen. The incisions can then be closed according to conventional methods. After implantation, the rate of drug delivery and/or the amount or dosage of drug that is delivered can be changed by controlling pump 30. For example, depending on how much pain the patient experiences, the rate of drug delivery and/or the amount or dosage of drug that is delivered can be changed. Similarly, the amount of fluid within the deformable body can be modulated post-operatively via, for example, filling valve 33.

While a number of embodiments have been described, the invention is not so limited. For example, to place the devices described herein from L4-5 and cephalad, a lateral approach can be used. The patient is placed in the lateral decubitus position, generally with the left side up, but right side up is also possible. A flank incision is made lateral to the paraspinal muscles and deep dissection is carried through the abdominal musculature. A finger is inserted down to the psoas muscle, and the peritoneum is dissected medially. Under fluoroscopic guidance, direct lateral incision is made and with the help of the finger in the flank incision, a guide member is directed down to the edge of the psoas muscle.

The guide member can have a variety of forms including a blunt tip rod or a guide assembly of an inner occluder and an outer tubular member fitted together having a tubular member lumen receiving the occluder. The occluder can have the form of a solid body member, such as an obturator, a stylet, or a guidewire, and the tubular member can have the form of a needle, a trocar, or a catheter.

The guide member is advanced through the psoas muscle to the edge of the disc and docked into the disc with a guidewire. This portion of the procedure is to be performed either with the patient awake or under general anesthesia with neural monitoring during penetration of the psoas. An outer tubular working cannula (e.g., approximately 6 mm diameter) is then placed over this initial guide member. This allows arthroscopic removal of the nucleus. In other embodiments, the guidewire is placed all the way across the disc and anchored to the outside of the annulus by a mechanical expansion device or a balloon. Shavers are then used around this initial guidewire to remove annular material.

After the nucleus is excised, a trial balloon is placed and inflated with radiographic dye. When adequate filling of the nucleus is confirmed, a nucleus replacement balloon (e.g., deformable body 28) is placed and inflated with a fluid such as saline. This balloon is attached via an elongated member (e.g., a non-expandable catheter) to a second fluid-filled subcutaneous reservoir. The skin is closed over the second subcutaneous reservoir. The amount of fluid within the placed device can now be regulated post-operatively and percutaneously. In some embodiments, the balloon also includes an outer permeable balloon that allows delivery of, for example, pain relieving medication, through a separate subcutaneous reservoir.

In other embodiments, such as for the L5-S1 disc, a trans-sacral approach is used. The patient is intubated. The anterior percutaneous pathway is performed with the patient in the prone position. An incision is made adjacent to the coccyx, and an elongated guide member is introduced through the skin incision and advanced against the anterior sacrum through the presacral space until the guide member distal end is located at the anterior target point (such as the junction of S1 and S2). The posterior viscera are pushed aside as the guide member is advanced through presacral space and axially aligned with the center of the disc.

The guide member can have a variety of forms including a blunt tip rod or a guide assembly of an inner occluder and an outer tubular member fitted together having a tubular member lumen receiving the occluder. The occluder can have the form of a solid body member, for example, an obturator, a stylet, or a guidewire, and the tubular member can have the form of a needle, a trocar, or a catheter.

The tissue surrounding the skin incision and the anterior presacral, percutaneous pathway through the presacral space can optionally be dilated to form an enlarged diameter presacral percutaneous tract surrounding a guidewire or tubular member and/or to accommodate the insertion of a tract sheath over the guidewire. Dilation can be accomplished manually or by use of one or more dilators, dilatation balloon catheters, or any tubular devices fitted over a previously extended tubular member or guidewire.

In a posterior approach, the posterior sacrum is exposed and a laminectomy is performed at S2. The posterior percutaneous tract is formed using conventional procedures and percutaneous tract tool sets. A curved axial bore is then made upwardly through S2, S1.

Thus, access is provided to anterior and posterior target points of the anterior or posterior sacrum preparatory to forming anterior or posterior bores that extend in the cephalad direction through the sacrum. The anterior or posterior bores can be employed to introduce instruments for removal of the nucleus and placement of the nuclear replacement device. An arthroscopic or mechanical shaver is placed through the cannula and advanced through the bore-hole in the sacrum and guided with fluoroscopic guidance to the L5-S1 disc.

After the nucleus is excised, a trial balloon is placed and inflated with radiographic dye. When adequate filling of the nucleus is confirmed, a nuclear replacement balloon (e.g., deformable body 28) is placed between the vertebras and inflated with a fluid such as saline. This balloon is attached via an elongated member (e.g., a non-expandable catheter) to a second fluid-filled subcutaneous reservoir (e.g., like fluid reservoir 31). The skin is closed over the second subcutaneous reservoir. The pressure within the placed device can now be regulated post-operatively and percutaneously (e.g., via a filling valve). In some embodiments, the balloon also includes an outer permeable balloon that allows delivery of, for example, pain relieving medication, through a separate subcutaneous reservoir.

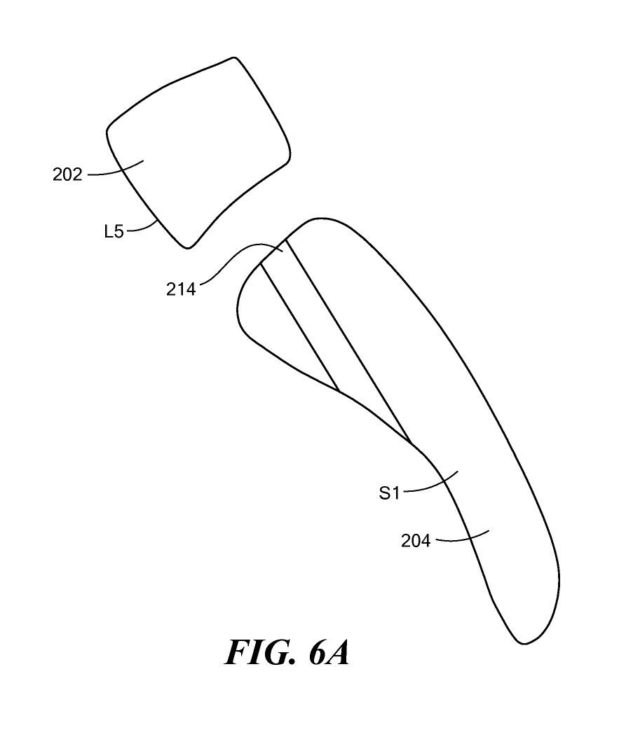

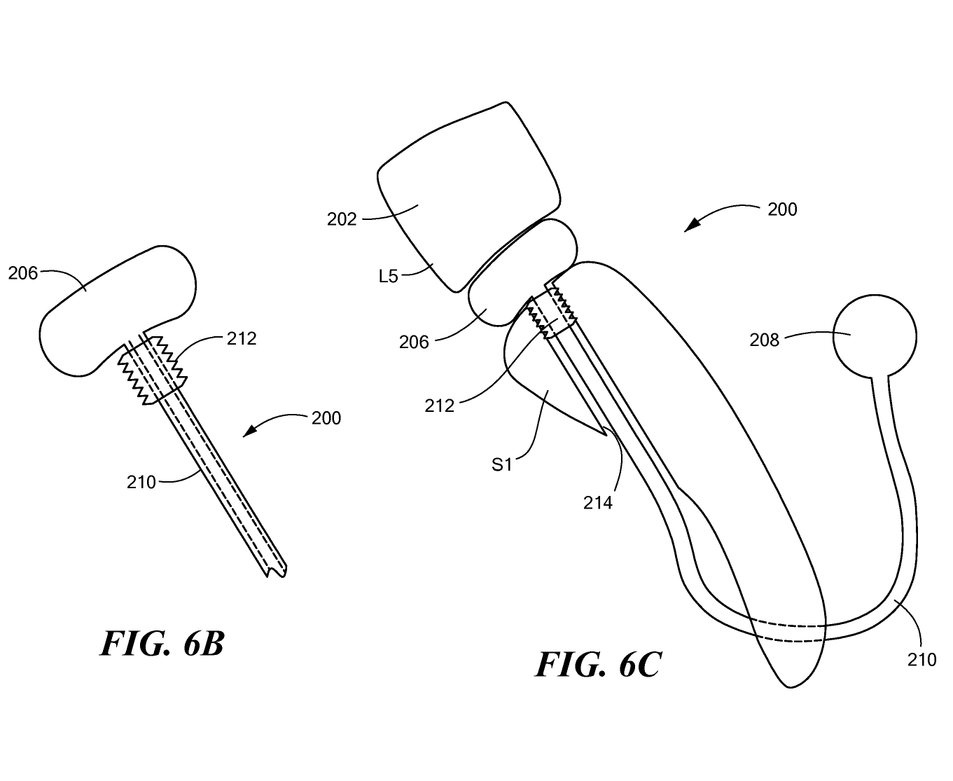

FIGS. 6A, 6B and 6C show a medical device 200 capable of being implanted between the L5 disc 202 and the S1 disc 204. As shown, device 200 includes a deformable body 206 (e.g., a balloon) capable of being implanted between discs 202, 204, a subcutaneous pump 208, and a hollow catheter 210 that provide fluid communication between an interior of the deformable body and the pump. While not shown for clarity, device 200 can include a fluid reservoir (e.g., like reservoir 31) having an interior volume in fluid communication with an interior volume of deformable body 206 to control the amount of fluid in the deformable body (e.g., via a subcutaneous filling valve). Catheter 210 can be formed of a flexible but non-expandable tubing material, such as a polymer. Deformable body 206 is configured to deliver a drug from pump 208 directly to the space between discs 202, 204. Examples of deformable body 206 are described above and below (e.g., deformable body 28, 62, 80). Similarly, pump 208 can be any of the pumps or reservoirs described herein (e.g., an intrathecal pump). Medical device 200 further includes an anchor such as a cannulated or hollow metal screw 212, configured to engage with (as shown, slidably receive) catheter 210 and to secure deformable body 206 at a selected implant position. As shown, anchor screw 212 is further configured with S1 disc 204, which includes a knurled or grooved outer surface to enhance the grip between the screw and the disc.

Referring particularly to FIG. 6C, device 200 can be implanted by forming a channel 214 in S1 disc 204, and securing screw 212 in the channel. Catheter 210 is secured to screw 212, and deformable body 206 is placed in the space between discs 202 and 204. Pump 208 may be implanted subcutaneously and is capable of delivering a drug through catheter 210 and to deformable body 206 to provide treatment.

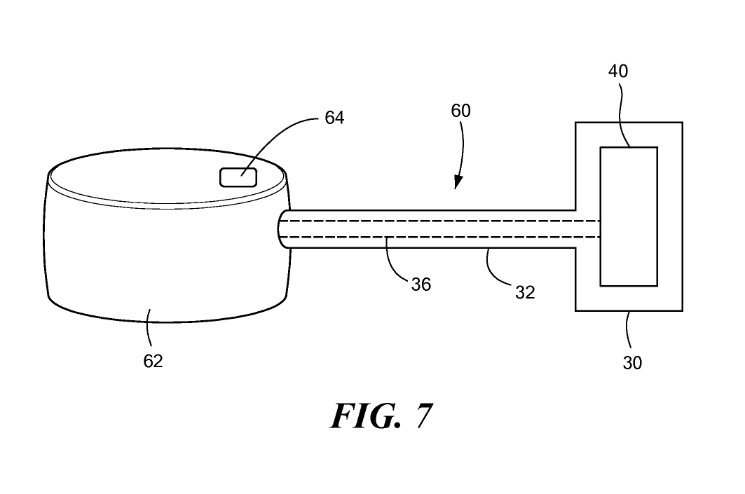

As another example, referring to FIG. 7, a medical device 60 includes a deformable body 62 having a controllable microvalve 64, or a plurality of microvalves. The embodiment depicted in FIG. 7 does not include a fluid reservoir (31) and a conduit (29), but in other embodiments the deformable body 62 includes these elements. Microvalve 64 serves as a gate for delivering the drug from the deformable body 62 into the spinal segment. The microvalve 64 is positioned in the deformable body so as to provide fluid communication between the deformable body and the spinal segment (when the valve is open). As depicted in FIG. 7, microvalve 64 may be positioned in the exterior wall of the deformable body 62. More particularly, the microvalve 64 may be positioned within an opening in the exterior wall so that the microvalve 64 is in fluid communication with a channel within the deformable body (e.g. the opening 34 and channel 38 of deformable body 28). The channel is further in fluid communication with a lumen 36 of a catheter 32 and a drug reservoir 40 having a pump 30. Such an arrangement allows for the drug to be delivered via pump 30 from the drug reservoir 40, through lumen 36, into the channel of deformable body 62, out microvalve 64 (when it is opened), and into desired areas, such as the spinal segment. Thus, the drug can be passed through microvalve 64, for example, to reduce pain.

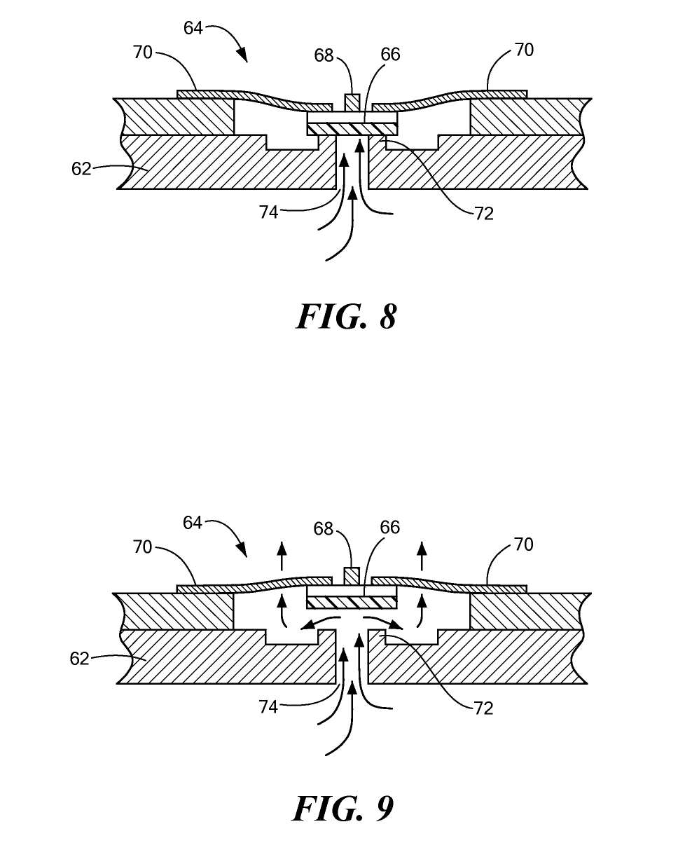

Referring to FIGS. 8 and 9, an example of microvalve 64 is shown. Microvalve 64 includes a poppet 66 that is connected to multiple fingers 68 and multiple cantilever arm segments 70. Fingers 68 are preformed with downward curves so that their ends exert a continuous downward bias force against the upper surface of poppet 66. Arm segments 70 include a shape memory alloy material (such as NiTi) that has been heat treated so that its memory shape, when heated through its phase change transition temperature, has the configuration shown in FIG. 8. Thus, in one configuration (FIG. 8), due to the downward bias force exerted by fingers 68, poppet 66 engages with a raised annulus 72 defined by deformable body 62 and prevents fluid from flowing past an opening 74 defined by the raised annulus. In another configuration (FIG. 9), when arm segments 70 are heated (e.g., 30 resistively heated by wires (not shown) connected to the arm segments) through the phase change transition temperature of the shape memory alloy, the arm segments move poppet 66 away from annulus 72, thereby allowing fluid and drug to flow past opening 74. When arm segments 70 are cooled below the transition temperature, the force exerted by fingers 68 moves arm segments 70 back to their previous shapes and poppet 66 back to sealing opening 74. Details of microvalves, including methods of making the valves, are described, for example, in Johnson et al., U.S. Pat. No. 5,325,880. In some embodiments, deformable body 62 has multiple valves 64, similar to deformable body 28 having multiple openings 34.

After the drug is depleted, medical device 60 can be replenished post-operatively via the drug reservoir 40 and catheter 32 in fluid communication with the interior volume of deformable body 62. Alternatively or additionally, a filling valve can be connected to deformable body 62, catheter 32, and/or reservoir 40 via a filler tube or catheter. The filling valve can be any device capable of being used to selectively open and close the filler tube to add fluid into deformable body 62. In some embodiments the filling valve may have a membrane that is penetrable to a needle and is self-sealing upon removal of the needle therefrom. Examples of filling valves include injection ports and infusion ports such as those used for the regular administration of medication (e.g., in chemotherapy) and/or regular blood withdrawal. Exemplary infusion ports include PORT-A-CATH from Pharmacia (Piscataway, N.J.); MEDI-PORT from Cormed (Cormed; Medina, N.Y.); INFUSE-A-PORT from Infusaid (Norwood, Mass.), and BARD PORT from Bard Access Systems (Salt Lake City, Utah). Other examples of filling valves include the PORT-CATH Systems (e.g. PORT-A-CATH Arterial System) available from Smith's Medical MD, Inc. (St. Paul, Minn.). In some embodiments, an implanted auxiliary supply of biocompatible fluid and/or drug connected to pump 30 can be used to refill medical device 60.

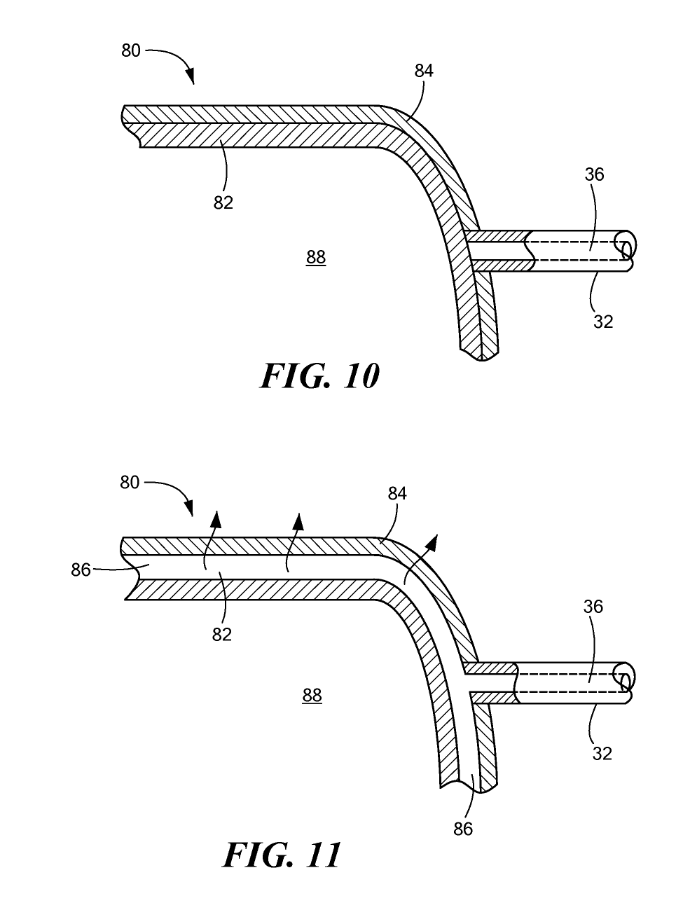

FIGS. 10 and 11 show an example of a medical device wherein the drug is delivered by passing the drug through a membrane. As shown, deformable body 80 includes an inner layer 82 and an outer layer 84 located outside the inner layer 82. The outer layer 84 may be coextensive with the inner layer 82, as depicted in FIG. 1. In other words, the inner layer 82 resides completely within the outer layer 84. The inner layer 82 forms an interior volume 88. The outer layer 84 and inner layer 82 form an exterior volume 86. Inner layer 82 includes (e.g., is formed of) an impermeable material (e.g., a polymer) through which the drug cannot pass. In some embodiments, inner layer 82 is reinforced (e.g., with a metallic mesh) to reduce creep. The interior volume 88 defined by inner layer 82 can be closed or in fluid communication with, for example, a subcutaneous fluid reservoir and/or filling valve so that the amount of fluid in the interior volume can be modulated post-operatively. Outer layer 84, on the other hand, includes (e.g., is formed of) a porous membrane through which the drug can pass. In some embodiments, the outer layer may include microvalves. The exterior volume 86 between layers 82, 84 is in fluid communication with lumen 36 of catheter 32. As a result, when the drug is delivered through lumen 36 and to deformable body 80, the drug can enter volume 86 and permeate through outer layer 84, thereby providing the desired treatment (FIG. 11). In some embodiments, selected areas of inner layer 82 and outer layer 84 are adhered together so that volume 86 forms channels that allow the drug to be delivered to selected areas of the implant site.

Yet, in other embodiments of the invention, the medical device does not include the inner and outer layers 82, 84. Instead, the medical device include a first balloon that acts as deformable body for support of the vertebras and a second balloon that includes a permeable membrane for releasing the drug. The balloons are configured so that they can both be implanted into the spinal segment (e.g., they are located alongside one another, or one on top of the other). Additionally, the two balloons may share a common structure or be formed from a single structure.

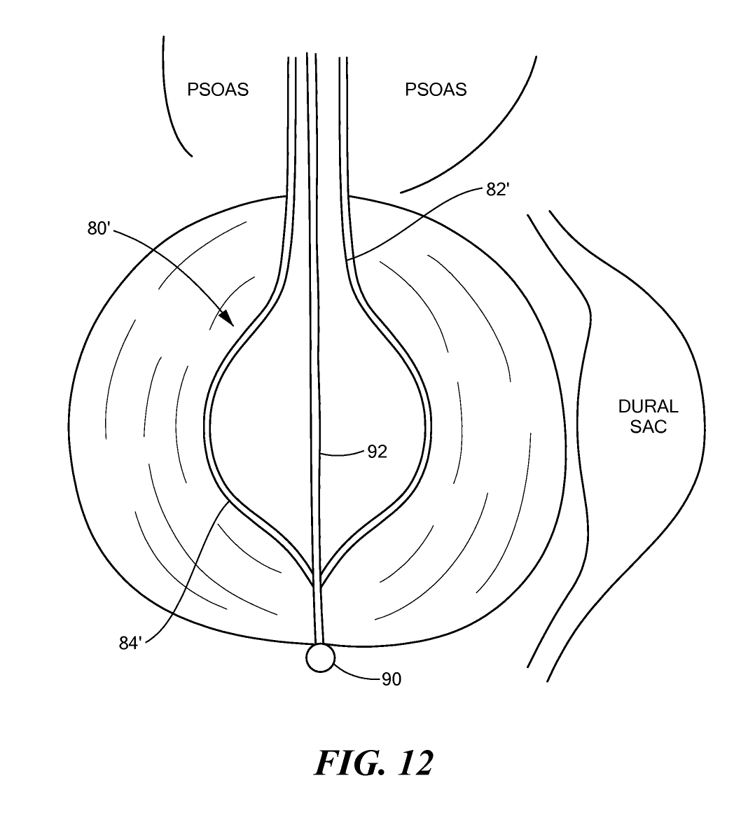

In other embodiments, deformable body 80 includes an additional balloon. FIG. 12 depicts a deformable body 80′ including an inner layer 82′, a coextensive outer layer 84′ and an anchoring balloon 90. Inner layer 82′ is generally as described above for layer 82. Layer 82′ defines a balloon that is fluid filled (e.g., with saline) to provide mechanical load bearing, with the amount of fluid within the balloon determining the height restoration within the disc. In some embodiments, inner layer 82′ is reinforced (e.g., with a metallic mesh) to reduce creep. The interior volume defined by inner layer 82′ can be closed or in fluid communication with, for example, a subcutaneous reservoir so that the fluid in the balloon can be modulated. Outer layer 84′ is generally as described above for layer 84. Layer 84′, which can be formed of a semi-permeable or porous material, defines a balloon that can be used to deliver a drug directly to the location where deformable body 80′ is implanted. The internal volume defined by outer layer 84′ is in fluid communication with a subcutaneous reservoir or a pump from which the drug is delivered. Anchoring balloon 90, which is connected to layers 82′, 84′ is used to secure body 80′ in a selected position, such as an outer annulus, to prevent unwanted movement of body 80′ after implantation. In effect, anchoring balloon 90 functions similarly to a toggle bolt that is used to secure an object to a hollow wall. The interior of anchoring balloon 90 is in fluid communication with a catheter 92 that extends through body 80′ and through which the anchoring balloon can be filled with a fluid, such as saline.

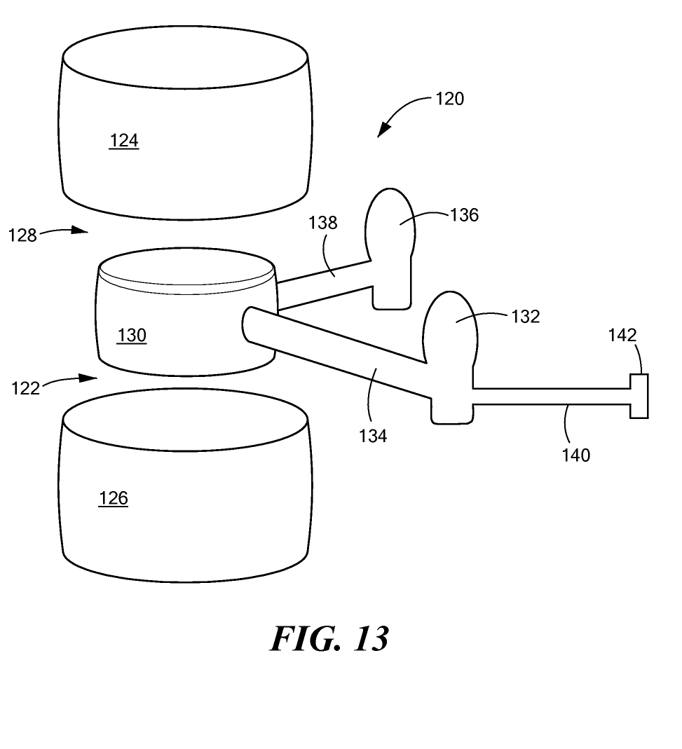

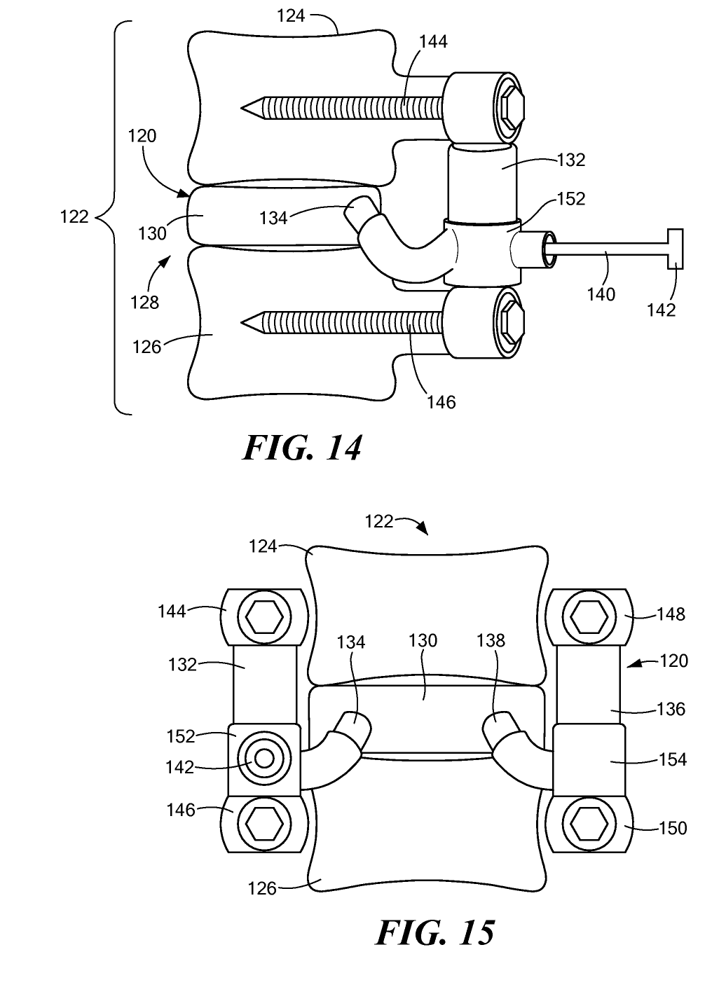

Referring to FIGS. 13, 14, and 15, a medical device system 120 is shown along a spinal segment 122 between a superior vertebra 124 and an inferior vertebra 126. Medical device system 120 includes an elongated member 128 having an expandable intradiscal portion 130, a first expandable extradiscal portion 132 in fluid communication with the intradiscal portion via a first hollow conduit 134, and a second extradiscal portion 136 in fluid communication with the intradiscal portion via a second hollow conduit 138. Elongated member 128 further includes a hollow filler tube 140 and a valve 142 for filling the elongated member with a fluid, such as saline, to a predetermined pressure. System 120 further includes multiple (as shown in FIGS. 14 and 15) pedicle screws 144, 146, 148, and 150 that attach the system to the spinal segment 122 (such as to the vertebras or spinal processes), and one or more (as shown, two) constraints 152 and 154 that surround portions of elongated portion 128 to prevent the portion(s) from expanding. As shown, elongated member 128 is secured to spinal segment 122 with intradiscal portion 130 positioned between vertebras 124 and 126 (for example, in place of a portion of an intervertebral disc), and extradiscal portions 132 and 136 positioned away from (as shown, posterior of) the intravertebral disc and/or vertebras.

In use, medical device system 120 is capable of mimicking an intervertebral disc to allow spinal segment 122 to move normally. In particular, system 120 uses the hydraulic pressure from the fluid filled in elongated member 128 to stabilize spinal segment 122 during motion. For example, when the patient bends or flexes forward, this movement can compress intradiscal portion 130, thereby transferring fluid by hydraulic pressure from the intradiscal portion to one or both of extradiscal portions 132 and 136 via conduits 134 and/or 138. One or both of extradiscal portions 132 and 136 can expand as a result of the additional fluid. In some embodiments, the extradiscal portion can be a piston. The expansion of extradiscal portions 132 and 136 can increase the forces of distraction of the vertebras or decrease the forces of distraction, for example, by controlling the manner in which the extradiscal portion(s) deform. When the patient bends or flexes backward, this movement can compress one or both of extradiscal portions 132 and/or 136, thereby transferring fluid by hydraulic pressure from the extradiscal portion(s) to intradiscal portion 130, which can expand as a result of the additional fluid. Similarly, when the patient rotates or bends laterally, fluid from one of extradiscal portions 132 or 136 can flow to and expand intradiscal portion 130 and/or the other extradiscal portion. Thus, medical device system 120 is capable of allowing spinal segment 122, such as a lumbar spinal segment, to move, for example, flex, rotate, and/or bend, relatively naturally while still maintaining mechanical integrity and stability.

Medical device system 120 can further include features described above for drug delivery. The intradiscal portion may be any of the deformable bodies described above (e.g., 28, 62, 206, 80). For example, in one embodiment, one or more drug reservoirs (e.g., associated with a pump) containing a drug can be placed in fluid communication via a hollow catheter(s) with one or more openings and channels formed in intradiscal portion 130 (e.g., the deformable body 28 depicted in FIGS. 1-4). In another embodiment, intradiscal portion 130 can include one or more microvalves. (e.g., the deformable body 62 depicted in FIGS. 7-9). In other embodiments, intradiscal portion 130 may include an inner layer that is impermeable to a drug, and an outer layer that is permeable to the drug (e.g., FIGS. 10, 11 and 12). Other embodiments of medical device systems that may be combined with the features described above for drug delivery are described in Raiszadeh, U.S. Application Publication No. 2006/0085074, which discloses, in more detail, medical device systems 120 and methods of implanting the systems.

As used herein, intradiscal portion 130 is a portion that is generally configured to be placed, wholly or partially, between two vertebras. Intradiscal portion 130 can be configured to occupy an intradiscal space, or the volume previously occupied by an intervertebral disc, between the vertebras. Intradiscal portion 130 can wholly or partially occupy the intradiscal space (e.g., the nucleus and annulus of the intradiscal space). In comparison, extradiscal portions 132 and 136 are generally configured not to be placed between two vertebras. In some embodiments, they are configured to be placed adjacent to the posterior facet joints. Extradiscal portions 132 and 136 can have various configurations (e.g., generally cylindrical, or generally oval). Intradiscal portion 130 and extradiscal portions 132 and 136 are all capable of expanding or compressing as a function of external compression forces and internal fluid pressure.

All references, such as patents, patent applications, and publications, referred to above are incorporated by reference in their entirety.

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A medical device for delivering a drug, the device comprising:
a deformable body configured to be implanted between a first vertebra and a second vertebra for providing shock absorption and stabilization of the vertebras, wherein the deformable body comprises:
an impermeable inner layer forming an interior volume;
an outer layer at least a portion of which is permeable, wherein the outer layer is at least partially outside the inner layer and forms an exterior volume between the inner and outer layers; and
a drug reservoir connected to the deformable body and in fluid communication with the exterior volume, wherein the medical device is capable of delivering the drug from the reservoir into the exterior volume and releasing the drug through the permeable portion of the outer layer.

2. A medical device according to claim 1, wherein at least a portion of the outer layer is a porous membrane.

3. A medical device according to claim 1, wherein the outer layer includes a microvalve.

4. A medical device according to claim 1, further comprising:
a catheter having a lumen, wherein the catheter connects the drug reservoir to the deformable body and the lumen is configured to deliver a drug from the drug reservoir to the exterior volume.

5. A medical device according to claim 1, wherein the outer layer is coextensive with the inner layer.

6. A medical device according to claim 1, further comprising:
a pump in fluid communication with the exterior volume for delivering the drug from the drug reservoir to the exterior volume.

7. A medical device according to claim 6, wherein the pump is configured to be implanted subcutaneously and is remotely controllable.

8. A medical device according to claim 1, further comprising:
a filling valve for modulating the drug in the drug reservoir.

9. A medical device according to claim 8, wherein the filling valve is an infusion port.

10. A medical device according to claim 1, wherein portions of the outer layer and inner layer are adhered together in order to form channels that allow drug delivery to selected areas of the implant site.

11. A medical device according to claim 1, wherein the inner layer is reinforced with a mesh material.

12. A medical device according to claim 1, further comprising:
a filling valve located outside the deformable body and in fluid communication with the interior volume, wherein the filling valve is configured to allow for post-operative addition or removal of fluid in the interior volume.

13. A medical device according to claim 12, wherein the filling valve is an infusion port.

14. A medical device according to claim 1, further comprising:
a reservoir for modulation of liquid within the interior volume, wherein the reservoir is connected to the deformable body and in fluid communication with the interior volume.

15. A medical device according to claim 1, further comprising:
an extradiscal portion spaced from the deformable body, wherein the extradiscal portion is connected to the deformable body and is in fluid communication with the interior volume of the deformable body.

16. A medical device according to claim 15, wherein the extradiscal portion is expandable.

17. A medical device according to claim 16, further comprising:
a first connector for attaching the extradiscal portion to a first portion of a spinal segment.

18. A medical device according to claim 17, further comprising:
a second connector for attaching the extradiscal portion to a second portion of the spinal segment.

19. A medical device according to claim 17, wherein the first portion of the spinal segment is the first vertebra.

20. A medical device according to claim 18, wherein the second portion of the spinal segment is the second vertebra

21. A medical device according to claim 17, wherein the first portion of the spinal segment is a first spinous process.

22. A medical device according to claim 18, wherein the second portion of the spinal segment is a second spinous process.

23. A medical device according to claim 17, wherein the first connector is a pedicle screw.

24. A medical device according to claim 18, wherein the extradiscal portion is configured to connect to the first and second spinal segments so that movement of the spinal segments and pressure on the intradiscal portion applies hydraulic pressure to the extradiscal portion.

25. A medical device according to claim 24, wherein the extradiscal portion includes a piston.

26. A medical device for delivery of a drug, the device comprising:
a deformable body configured to be implanted between a first vertebra and a second vertebra for providing shock absorption and stabilization of the vertebras, wherein the deformable body comprises a deformable exterior wall and a deformable interior wall forming an interior volume;
a channel formed between the exterior and interior walls, wherein the channel is in fluid communication with an opening formed in the exterior wall of the deformable body; and
a drug reservoir connected to the deformable body and in fluid communication with the channel, wherein the medical device is capable of delivering the drug from the drug reservoir into the channel and releasing the drug through the opening in the exterior wall.

27. A medical device according to claim 26, wherein the medical device includes a plurality of openings.

28. A medical device according to claim 27, wherein the medical device includes a plurality of channels.

29. A medical device according to claim 26, wherein openings have different dimensions.

30. A medical device according to claim 28, wherein the channels have different dimensions.

31. A medical device according to claim 26, wherein the medical device includes at least one valve in fluid communication with the channel for modulating the release of the drug through at least one opening.

32. A medical device according to claim 31, wherein the valve is a microvalve located in the exterior wall.

33. A medical device according to claim 26, further comprising:
a catheter having a lumen, wherein the catheter connects the drug reservoir to the deformable body and the lumen is configured to deliver a drug from the drug reservoir through the opening.

34. A medical device according to claim 26, further comprising:
a pump in fluid communication with the channel for delivering the drug from the drug reservoir to the channel and through the opening.

35. A medical device according to claim 34, wherein the pump is configured to be implanted subcutaneously and is remotely controllable.

36. A medical device according to claim 26, further comprising:
a filling valve for modulating the drug in the drug reservoir.

37. A medical device according to claim 36, wherein the filling valve is an infusion port.

38. A medical device according to claim 26, further comprising:
a filling valve located outside the deformable body and in fluid communication with the interior volume, wherein the filling valve is configured to allow for post-operative addition or removal of fluid in the interior volume.

39. A medical device according to claim 38, wherein the filling valve is an infusion port.

40. A medical device according to claim 26, further comprising:
a reservoir for modulation of liquid within the interior volume, wherein the reservoir is connected to the deformable body and in fluid communication with the interior volume.

41. A medical device according to claim 26, further comprising:
an extradiscal portion spaced from the deformable body, wherein the extradiscal portion is connected to the deformable body and is in fluid communication with the interior volume of the deformable body.

42. A medical device according to claim 41, wherein the extradiscal portion is expandable.

43. A medical device according to claim 41, further comprising:
a first connector for attaching the extradiscal portion to a first portion of a spinal segment.

44. A medical device according to claim 43, further comprising:
a second connector for attaching the extradiscal portion to a second portion of the spinal segment.

45. A medical device according to claim 43, wherein the first portion of the spinal segment is the first vertebra.

46. A medical device according to claim 44, wherein the second portion of the spinal segment is the second vertebra

47. A medical device according to claim 43, wherein the first portion of the spinal segment is a first spinous process.

48. A medical device according to claim 44, wherein the second portion of the spinal segment is a second spinous process.

49. A medical device according to claim 43, wherein the first connector is a pedicle screw.

50. A medical device according to claim 44, wherein the extradiscal portion is configured to connect to the first and second spinal segments so that movement of the spinal segments and pressure on the intradiscal portion applies hydraulic pressure to the extradiscal portion.

51. A medical device according to claim 50, wherein the extradiscal portion includes a piston.

52. A method for delivering a drug, the method comprising:
providing a medical device having a deformable body configured to be implanted between a first vertebra and a second vertebra for providing shock absorption and stabilization of the vertebras and a drug reservoir connected to the deformable body, wherein the device is capable of delivering a drug from the drug reservoir into the deformable body and releasing the drug through the deformable body into an implant site;
positioning the deformable body between two vertebras;
after positioning the deformable body between the two vertebras, adding biocompatible fluid into the deformable body;
positioning the drug reservoir in the body;
adding a drug to the drug reservoir; and
retaining in the body post-operatively the drug reservoir and the deformable body between the two vertebras.

53. A method according to claim 52, further comprising:
delivering the drug from the drug reservoir to the deformable body.

54. A method according to claim 52, further comprising:
modulating the biocompatible fluid in the deformable body post-operatively.

55. A method according to claim 52, further comprising:
modulating the drug in the drug reservoir post-operatively.

56. A method according to claim 53, further comprising:
changing a rate of drug delivery.

57. A method according to claim 56, wherein the rate of delivery is a function of pain experienced by the patient.

58. A method according to claim 52, wherein the drug is an anesthetic.

59. A method according to claim 53, further comprising:
pumping the drug from the reservoir to the deformable body.

60. A method according to claim 59, wherein pumping the drug to the deformable body is controlled remotely post-operatively.

61. A method according to claim 52, wherein the deformable body and the drug reservoir are positioned in the body from a posterior approach.

62. A method according to claim 52, further comprising:
removing at least a portion of a disc between the two vertebras.

63. A method according to claim 54, wherein the biocompatible fluid is modulated from a posterior approach through a filling valve in fluid communication with the deformable body.

64. A method according to claim 52, wherein the medical device further comprises an expandable extradiscal portion in fluid communication with the deformable body, and further comprising positioning the expandable extradiscal portion spaced from the vertebras.

65. A method according to claim 64, wherein the expandable extradiscal portion is positioned posterior of the vertebras.

66. A method according to claim 52 further comprising:
expanding a test balloon between the vertebras.

67. A method according to claim 66 further comprising:
adding a contrast agent into the test balloon.
